(19) United States
(12) Patent Application Publication
(10) Pub. No.: US 2026/0207742 A1
(43) Pub. Date: Jul. 23, 2026

(54) PHARMACEUTICAL COMBINATIONS FOR TREATING TUMOR COMPRISING ANTI-CD19 ANTIBODY AND NATURAL KILLER CELL

(71) Applicants: GC CELL CORPORATION, SEOUL (KR); Incyte Corporation, Wilmington, DE (US)

(72) Inventors: Jung Hyun Her, Gyeonggi-do (KR); Mi Young Jung, Gyeonggi-do (KR); Su Hyun Gwon, Gyeonggi-do (KR); Ho Yong Lim, Gyeonggi-do (KR); Sung Yoo Cho, Gyeonggi-do (KR); Sung Yong Won, Gyeonggi-do (KR); Yu Kyeong Hwang, Gyeonggi-do (KR); Jan Endell, Planegg (DE); Rainer Boxhammer, Planegg (DE)

(21) Appl. No.: 19/389,356

(22) Filed: Nov. 14, 2025

Related U.S. Application Data
(63) Continuation of application No. 17/275,292, filed on Sep. 5, 2019, now Pat. No. 12,491,244.

(30) Foreign Application Priority Data
Sep. 12, 2018 (KR) 10-2018-0109093

(51) Int. Cl. A61K 39/395; A61K 40/15; A61K 40/42; A61P 35/00; C07K 16/28; C12N 5/0783

(52) U.S. Cl. CPC A61K 39/3955; A61K 40/15; A61K 40/42; A61P 35/00; C07K 16/2896; C12N 5/0646; A61K 2239/31; A61K 2239/38; A61K 2239/48

(57) ABSTRACT

Provided is a pharmaceutical combination comprising an antibody specific for CD19 and a natural killer cell, and a treatment method using the same. Such a pharmaceutical combination is capable of exhibiting synergistic therapeutic effects on a malignant tumor of B-cell origin such as non-Hodgkin's lymphoma, chronic lymphocytic leukemia, and/or acute lymphoblastic leukemia.

### CROSS-REFERENCE TO RELATED APPLICATIONS

This application is a continuation application of and claims the benefit of priority to U.S. application Ser. No. 17/275,292, filed on Mar. 11, 2021, which is a national stage of International Application No. PCT/KR2019/011474, filed on Sep. 5, 2019, which claims the benefit of priority from KR 10-2018-0109093, filed Sep. 12, 2018, the contents of which are hereby incorporated by reference.

### SEQUENCE LISTING

This application contains a Sequence Listing that has been submitted electronically as an XML file named “20443-0686002_SL_ST26.XML.” The XML file, created on Nov. 5, 2025, is 11,769 bytes in size. The material in the XML file is hereby incorporated by reference in its entirety.

### TECHNICAL FIELD

An embodiment of the present invention is directed to a pharmaceutical combination comprising an anti-CD19 antibody and a natural killer cell, which has synergistic therapeutic effects on a malignant tumor of B cell origin such as non-Hodgkin's lymphoma, chronic lymphocytic leukemia (CLL), and/or acute lymphoblastic leukemia (ALL).

### BACKGROUND ART

Natural killer cells (NK cells) are known to be lymphoid cells that recognize a target in a non-MHC-restricted manner unlike T cells and play an important role in innate immune responses. The natural killer cells can exert anti-viral and anti-cancer efficacies. Specifically, the natural killer cells play a role of directly killing a malignant tumor, or of inducing dendritic cell activities or tumor-specific cytotoxic T lymphocytes (CTLs) to eliminate abnormal cells that have developed tumors or are developing tumors. In addition, regarding the anti-cancer efficacies of the natural killer cells, it has been proven that use of allogeneic natural killer cells having mismatch with killer cell immunoglobulin-like receptor (KIR)-ligand pairs in the treatment of cancer patients is much more efficacious and safer than use of autologous natural killer cells.

On the other hand, human CD19 molecules are cell surface receptors expressed on surfaces of human B cells, for example, pre-B cells, immature B cells at an early stage of development, mature B cells, and malignant B cells. In fact, most of B-cell lineage malignant tumors express CD19, including non-Hodgkin's lymphoma, chronic lymphocytic leukemia (CLL), and acute lymphoblastic leukemia (ALL). Therefore, anti-CD19 antibodies can bind to CD19 antigens as targets, and thus can be used in a therapeutic immunotherapy of the above-mentioned malignant tumors of B cell origin.

Despite recent discoveries and developments of several anti-cancer agents, due to poor prognosis for many types of cancers including CD19-expressing tumors, there is still a need for an improved method or therapeutic agent for treating such types of cancers. Accordingly, the present inventors have confirmed that combined administration of a natural killer cell and an antibody specific for CD19 has synergistic effects on the treatment of malignant lymphomas of B cell origin, and have completed the present invention.

### CITATION LIST

### Non-Patent Literature

Nadler et al., J. Immunol., 131: 244-250 (1983)

### SUMMARY OF INVENTION

### Technical Problem

An object of the present invention is to provide a combined therapy using an antibody specific for CD19 and a natural killer cell, or a combination thereof.

### Solution to Problem

In order to achieve the above object, an embodiment of the present invention can provide a pharmaceutical combination for the treatment of cancer, comprising an antibody specific for CD19 and a natural killer cell (NK cell).

The antibody may have a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3).

The antibody may have a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

The antibody may have a heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3), and a light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

The antibody may comprise a heavy chain variable region of EVQLVESGGGLVKPGGSLKLSCAASGYTFTSYVMHWVRQAPGKGLEWIGYINPYND GTKYNEKFQGRVTISSDKSISTAYMELSSLRSEDTAMYYCARGTYYYGTRVFDYWGQ GTLVTVSS (SEQ ID NO: 8) and a light chain variable region of DIVMTQSPATLSLSPGERATLSCRSSKSLQNVNGNTYLYWFQQKPGQSPQLLIYRMSN LNSGVPDRFSGSGSGTEFTLTISSLEPEDFAVYYCMQHLEYPITFGAGTKLEIK (SEQ ID NO: 9).

The antibody may comprise a heavy chain constant region of ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPE LLGGPDVFLFPPKPKDTLMISRTPEVTCVVVDVSHEDPEVQFNWYVDGVEVHNAKT KPREEQFNSTFRVVSVLTVVHQDWLNGKEYKCKVSNKALPAPEEKTISKTKGQPREP QVYTLPPSREEMTKNQVSLTCLVKGFYPSDIAVEWESNGQPENNYKTTPPMLDSDGS FFLYSKLTVDKSRWQQGNVFSCSVMHEALHNHYTQKSLSLSPGK (SEQ ID NO: 10).

The antibody may comprise a light chain constant region of RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 11).

The NK cell may be prepared by a method comprising step (i) of performing a stationary culture of NK cells in a culture solution containing anti-CD3 antibodies, cytokines, and feeder cells, to stimulate intercellular contacts, step (ii) of adding cytokines, anti-CD3 antibodies, and feeder cells thereto for re-stimulation and performing a stationary culture again to induce intracellular contacts, and step (iii) of adding a medium containing cytokines to the cells and performing a stationary or suspension culture while constantly maintaining a concentration of the cells and a concentration of the cytokines.

The NK cell may be prepared by a method comprising a step of co-culturing CD4(+) T cells that have been isolated ex vivo or CD4(+) T cells that have been expansion-cultured ex vivo, as feeder cells, and seed cells.

The CD4(+) T cells may be CD4(+)/CD1(−) T cell lines.

The CD4(+)/CD1(−) T cell lines may be H9 or HuT78 cell lines.

The seed cells may be one or more selected from the group consisting of peripheral blood-derived cells, peripheral blood leukocyte cells, peripheral blood mononuclear cells (PBMCs), umbilical cord blood leukocyte cells, umbilical cord blood mononuclear cells, natural killer cells derived from stem cells, enriched natural killer cells, and isolated natural killer cells.

Further, the pharmaceutical combination may further comprise albumin.

The cancer may be at least one selected from the group consisting of gastric cancer, liver cancer, lung cancer, colorectal cancer, breast cancer, prostate cancer, ovarian cancer, pancreatic cancer, cervical cancer, thyroid cancer, laryngeal cancer, acute lymphoblastic leukemia, brain tumor, neuroblastoma, retinoblastoma, head and neck cancer, salivary gland cancer, and lymphoma.

The lymphoma may be a malignant tumor of B cell origin.

The malignant tumor of B cell origin may be at least one selected from the group consisting of non-Hodgkin's lymphoma, chronic lymphocytic leukemia, and acute lymphoblastic leukemia.

The antibody specific for CD19 and the NK cell may be administered in a separate manner.

The antibody specific for CD19 and the NK cell may be administered in a simultaneous manner.

Another embodiment of the present invention can provide a kit for the treatment of cancer, comprising the above-described combination.

Still another embodiment of the present invention can provide a use of the above-described pharmaceutical combination for preparing a medicament for the treatment of cancer.

Further, yet another embodiment of the present invention can provide a method for the treatment of cancer, comprising a step of administering, to a subject, the above-mentioned antibody specific for CD19 and the above-mentioned NK cell in combination.

### Advantageous Effects of Invention

The pharmaceutical combination comprising an antibody specific for CD19 and a natural killer cell, according to the embodiment of the present invention, is useful for the treatment of cancer, in particular, a B-cell malignant tumor such as non-Hodgkin's lymphoma, chronic lymphocytic leukemia, and/or acute lymphocytic leukemia. In particular, the natural killer cell is produced in a clinically friendly manner as compared with conventional methods so as to have high cell-killing efficacy, cell survival rate, and long-term storability, and is capable of exhibiting synergistic effects on the treatment of tumor in a case of being administered in combination with a certain antibody specific for CD19.

### BRIEF DESCRIPTION OF DRAWINGS

FIGS. 1a to 1d show results obtained by culturing Burkitt's B-cell lymphomas, Raji and Ramos, with various concentrations of monoclonal antibodies in the presence of NK cells.

FIG. 2 shows results obtained by evaluating suitability of Ramos cell lines in an SCID mice model.

FIG. 3 shows results obtained by administering MOR208 at different concentrations in an SCID mice model group into which Ramos cells had been intravenously injected.

FIG. 4 shows results obtained by administering MG4101 at different concentrations in an SCID mice model group into which Ramos cells had been intravenously injected.

FIG. 5 shows results obtained by administering a control, MOR208, MG4101, and “MOR208/MG4101” in an SCID mice model group into which Ramos cells had been intravenously injected.

### DESCRIPTION OF EMBODIMENTS

Despite a possibility that natural killer cells can be applied as therapeutic agents for various diseases, it has not been easily accessed due to several limitations such as a rather restricted number of natural killer cells in peripheral blood, difficulties associated with good manufacturing practice (GMP)-compliant large-scale production for cytolytic natural killer cells, and natural killer cells having to be activated to induce natural killer cell-mediated killing.

Recently, the present inventors have established a simple and efficient method for large-scale proliferation and activation of natural killer cells derived from healthy donors under GMP conditions (KR10-1644984 B1). In addition, natural killer cells, which have been proliferated ex vivo through a method for large-scale culture of NK cells from umbilical cord blood mononuclear cells and highly activated, were produced under GMP conditions, and exhibited strong anti-cancer activities in vitro and in vivo in preclinical studies.

On the other hand, treatment methods using a monoclonal antibody specific to an epitope molecule expressed in cancer cells have been known as the most successful cancer immunotherapies to date. One of action mechanisms thereof is natural killer cell-mediated antibody-dependent cellular cytotoxicity (ADCC). Since the natural killer cell proliferated according to an embodiment of the present invention expressed high levels of CD16 which is an Fc receptor that mediates ADCC, the present inventors have made an attempt to check that anti-cancer activities thereof against B cell lymphoma can be enhanced in the presence of an anti-CD19 antibody.

Surprisingly, the present inventors have found that, in a case of administering a certain antibody specific for CD19 and a natural killer cell in combination, synergistic effects are exhibited in vitro and in vivo on direct death of a human B cell malignant tumor through ADCC, as compared with a case of administering the antibody or natural killer cell alone.

One aspect of the present invention provides a pharmaceutical combination for the treatment of cancer, comprising an antibody specific for CD19 and a natural killer cell.

As used herein, the term “CD19” refers to a protein known as CD19 having the following synonyms: B4, B-lymphocyte antigen CD19, B-lymphocyte surface antigen B4, CVID3, differentiation antigen CD19, MGC12802, and T-cell surface antigen Leu-12. The CD19 may be a human-derived CD19, and the human CD19 may comprise an amino acid sequence of SEQ ID NO: 7. The CD19 is expressed by cells and tissues with various diseases and pathologies, including most of B cell malignant tumors, and on normal B cells.

Further, as used herein, the term “antibody” refers to a monoclonal antibody including any isotype such as IgG, IgM, IgA, IgD, and IgE. The IgG antibody consists of two identical heavy chains and two identical light chains, linked by disulfide bonds. Each of the heavy chains and light chains comprises a constant region and a variable region. Each variable region comprises three segments called “complementarity-determining regions (CDRs)” or “hypervariable regions” that are primarily responsible for binding to an epitope of an antigen. These are sequentially numbered from an N-terminus and are referred to as CDR1, CDR2, and CDR3. A more highly conserved portion outside the CDRs in the variable region is called a “framework region”.

That is, an anti-CD19 antibody is an antibody that specifically binds to a CD19 antigen, and may comprise not only a complete antibody form but also an antigen-binding fragment thereof.

Examples of the antibody specific for CD19 according to an embodiment of the present invention may include those described in U.S. Ser. No. 12/377,251 (Xencor); WO 2005/012493, WO 2010/053716 (Immunomedics); WO 2007/002223 (Medarex); WO 2008/022152 (Xencor); WO 2008/031056 (Medimmune); WO 2007/076950 (Merck Patent GmbH); WO 2009/052431 (Seattle Genetics); and WO 2010/095031 (Glenmark Pharmaceuticals).

Further, the antibody specific for CD19 according to the embodiment of the present invention may comprise the heavy chain variable region comprising an HCDR1 region of SYVMH (SEQ ID NO: 1), an HCDR2 region of NPYNDG (SEQ ID NO: 2), and an HCDR3 region of GTYYYGTRVFDY (SEQ ID NO: 3). Besides, the antibody specific for CD19 of the present invention may comprise the light chain variable region comprising an LCDR1 region of RSSKSLQNVNGNTYLY (SEQ ID NO: 4), an LCDR2 region of RMSNLNS (SEQ ID NO: 5), and an LCDR3 region of MQHLEYPIT (SEQ ID NO: 6).

An antibody specific for CD19 according to another embodiment of the present invention may include an antibody that cross-competes with the antibody of the present invention as described above.

As used herein, the term “cross-compete” means an ability of an antibody or other binding agent to interfere with binding of another antibody or binding agent to CD19 in a standard competitive binding assay. The ability or extent to which the antibody or other binding agent is capable of interfering with the binding of another antibody or binding agent to CD19, and thus whether it can be referred to as cross-competition according to the invention, can be determined using the standard competitive binding assay.

Besides, the antibody specific for CD19 according to another embodiment of the present invention may include an antibody binding to the same epitope as the antibody of the present invention as described above.

Further, the antibody may comprise a heavy chain variable region of EVQLVESGGGLVKPGGSLKLSCAASGYTFTSYVMHWVRQAPGKGLEWIGYINPYND GTKYNEKFQGRVTISSDKSISTAYMELSSLRSEDTAMYYCARGTYYYGTRVFDYWGQ GTLVTVSS (SEQ ID NO: 8) and a light chain variable region of DIVMTQSPATLSLSPGERATLSCRSSKSLQNVNGNTYLYWFQQKPGQSPQLLIYRMSN LNSGVPDRFSGSGSGTEFTLTISSLEPEDFAVYYCMQHLEYPITFGAGTKLEIK (SEQ ID NO: 9). In addition, the antibody may comprise a heavy chain constant region of ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPE LLGGPDVFLFPPKPKDTLMISRTPEVTCVVVDVSHEDPEVQFNWYVDGVEVHNAKT KPREEQFNSTFRVVSVLTVVHQDWLNGKEYKCKVSNKALPAPEEKTISKTKGQPREP QVYTLPPSREEMTKNQVSLTCLVKGFYPSDIAVEWESNGQPENNYKTTPPMLDSDGS FFLYSKLTVDKSRWQQGNVFSCSVMHEALHNHYTQKSLSLSPGK (SEQ ID NO: 10). In addition, the antibody may comprise a light chain constant region of RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO: 11). The above described anti-CD19 antibody may be “MOR208”. The MOR208 antibody may be one disclosed in U.S. patent application Ser. No. 12/377,251.

As used herein, the term “natural killer cell (hereinafter referred to as NK cell) refers to a cytotoxic lymphocyte that plays an important role in the body's immune system. For purposes of the present invention, the NK cell may be an endogenous NK cell of a subject and/or an exogenous NK cell, specifically an isolated NK cell which is administered externally of the subject, but is not limited thereto. In addition, the NK cell can be obtained by purchase of a commercially available product, or according to conventional methods such as isolation from a subject or culture.

On the other hand, the NK cell contained in the pharmaceutical combination according to the embodiment of the present invention can be one prepared by stimulating mononuclear cells, from which T cells have been removed, with anti-CD3 antibodies and feeder cells in a medium containing cytokines, performing a stationary culture for several days to stimulate intracellular contacts, and performing a stationary or suspension culture of NK cells while constantly maintaining a concentration of the cells and a concentration of the cytokines.

In order to obtain an increased amount of NK cells, stimulation and stationary culture may be repeated before the subsequent stationary or suspension culture. The NK cell prepared by the method according to the present invention can be cultured ex vivo with high efficiency and high concentration while exhibiting high cell survival rate and cell-killing efficacy, and thus can be administered in combination with the anti-CD19 antibody to treat a malignant tumor of B cell origin.

As an example, the NK cell may be one prepared by a method comprising the following steps (i) to (iii).

Step (i) of performing a stationary culture of isolated NK cells in a culture solution containing anti-CD3 antibodies, cytokines, and feeder cells, to stimulate intercellular contacts; step (ii) of adding cytokines, anti-CD3 antibodies, and feeder cells thereto for re-stimulation and performing a stationary culture again to induce intracellular contacts; and step (iii) of adding a medium containing cytokines to the cells and performing a stationary or suspension culture while constantly maintaining a concentration of the cells and a concentration of the cytokines.

Here, the isolated NK cells may be obtained by isolating leukocyte cells and NK cells from human peripheral blood or umbilical cord blood.

Here, the stationary culture after the initial stimulation may be performed for about 2 to 15 days, specifically for 5 to 10 days, and the stationary culture after the re-stimulation may be performed for about 2 to 7 days, specifically for 3 to 5 days. In addition, after completion of the stationary cultures, a stationary or suspension culture may be performed in an incubator while constantly maintaining a concentration of the cytokines. In addition, in a case of preparing the NK cell, as albumin is added to a composition containing an NK cell, cell-killing efficacy and cell survival rate of the NK cell can be greatly raised. Specifically, the NK cell prepared by the above-described method can be denoted as “MG4101”. The method for preparing the NK cell according to an embodiment of the present invention and the NK cell may be those described in Korean Patent No. 10-1644984.

As used herein, the term “feeder cell (also referred to as culture-assisting cell)” refers to a cell that does not have an ability to proliferate by division but has a metabolic activity, thereby producing various metabolites to help proliferation of a target NK cell. As the feeder cells that can be used, animal cell lines into which genes have been introduced, peripheral blood leukocytes (PBLs) treated with various cytokines or compounds, self or non-self peripheral blood leukocytes (PBLs), T-cells, B-cells, or monocytes, or the like may be used, and, specifically, self peripheral blood mononuclear cells may be used, but not limited thereto.

Further, the self peripheral blood mononuclear cells used as the feeder cells can be inactivated to ensure safety. As an inactivation method, a conventional method known in the art can be used, and, for example, a gamma-ray irradiation method can be used. Such inactivated feeder cells include isolated T-cells. A proliferation method using the feeder cells as described above is a method of proliferating NK cells after pure isolation thereof, and has an advantage that only pure NK cells are continuously proliferated afterward.

As used herein, the term “anti-CD3 antibody” refers to an antibody that specifically binds to a CD3 antigen, which is a molecule group that binds to a T cell receptor (TCR) to form an antigen-recognition complex. A CD3 molecule binds to TCR and serves to transmit an antigen-recognition signal into a cell. The anti-CD3 antibody usable in the present invention is not limited as long as it is an antibody having properties of binding to CD3. For example, the anti-CD3 antibody can be selected from the group consisting of OKT3, UCHT1, and HIT3a, but is not limited thereto.

In the present invention, the cytokines that can be contained in a medium may be one or more selected from interleukins. For example, one or more selected from the group consisting of interleukin-2 (IL-2), interleukin-12 (IL-12), interleukin-15 (IL-15), interleukin-18 (IL-18), and interleukin-21 (IL-21) can be used, but not limited thereto.

Further, a concentration of the anti-CD3 antibodies in a medium, which is used for stationary culture and suspension culture, may be 0.1 to 1,000 ng/ml, 1 to 100 ng/ml, or 5 to 20 ng/ml, and a concentration of the cytokines in a medium may be 10 to 2,000 IU, 100 to 1,000 IU, or about 200 to 700 IU.

As used herein, the term “stimulation” means inducing proliferation of NK cells by adding the feeder cells or the like thereto, in which anti-CD3 antibodies may be used together. In addition, as used herein, the term “re-stimulation” means re-inducing proliferation of the NK cells by adding the feeder cells and/or the anti-CD3 antibodies again to a medium after a certain time of culture has elapsed.

As a medium for the preparation of the NK cell according to the embodiment of the present invention, a common medium for animal cell cultures such as CellGro medium (Cellgenix), AIM-V medium, RPMI 1640 medium, and X-VIVO 20 can be used.

In particular, in the preparation method of the NK cell, in order to constantly maintain a concentration of the cells and the cytokines during suspension culture, concentrations of the cytokines and cells in a medium can be measured at certain time intervals, and, depending on the measured values, a cytokine-containing medium may be provided to comply with the concentration of the cells and the cytokines.

Further, culture may be performed by adding, to the medium, serum or plasma, and an additional proliferation factor that supports proliferation of lymphocytes. Types of serum or plasma to be added to the medium are not particularly limited. Any commercially available animal-derived one can be used, and human-derived one which is derived from the same person can be used. For example, it is possible to add a combination of cytokines that proliferate lymphocytes from peripheral blood mononuclear cells, lectins that stimulate proliferation of lymphocytes, or the like.

In particular, as albumin is added to a composition containing the NK cell prepared by the method of the present invention, in terms of long-term storability of the NK cell, cell-killing efficacy and cell survival rate thereof can be greatly raised. An amount of albumin to be added is not particularly limited. Albumin may be contained, specifically, in a range of 0.1% to 5% by weight, and, more specifically, in a range of 0.5% to 2% by weight, within the entire composition.

Further, by applying a culture method that constantly maintains a concentration of cells for the preparation of NK cells, it is possible to prevent overgrowth of the cells, thereby maintaining the cells in an optimal state. In particular, even in a case of being thawed after freezing, the cells do not have impaired function, and can maintain high cell survival rate and cell-killing efficacy. Therefore, there is an advantage that storage and supply are easily done in a liquid or frozen storage form without additional processing.

On the other hand, the NK cell used in the present invention may be, as an example, one prepared by a method comprising a step of co-culturing CD4(+) T cells that have been isolated ex vivo or CD4(+) T cells that have been expansion-cultured ex vivo, as feeder cells, and seed cells.

Specifically, the CD4(+) T cells used in the present invention may be CD4(+)/CD1(−) T cell lines, and, more specifically, may be H9 or HuT78 cell lines.

As used herein, the term “seed cell” means a cell as a starting material for obtaining a target cell. The seed cells used in the present invention may be, but not limited to, one or more selected from the group consisting of peripheral blood-derived cells, peripheral blood leukocyte cells, peripheral blood mononuclear cells (PBMC), umbilical cord blood leukocyte cells, umbilical cord blood leukocyte cells, umbilical cord blood mononuclear cells, natural killer cells derived from stem cells, enriched natural killer cells, and isolated natural killer cells.

With respect to the preparation method of the NK cell according to the embodiment of the present invention and the NK cell, those may be the ones described in Korean Patent Nos. 10-1697473 or 10-1799986.

Such a preparation method of the NK cell is capable of not only selectively proliferating only NK cells at a large scale from a small amount of seed cells, but also allowing maintenance of high killing efficacy thereof. Therefore, due to containing the NK cell produced by such a method thus has high cell survival rate and cell-killing efficacy, and a certain anti-CD19 antibody, the pharmaceutical combination according to an embodiment of the present invention exhibits quite excellent cancer therapeutic effects.

Further, as used herein, the term “combination” means a composition in which more than one effective ingredient, for example, effective ingredients such as an antibody and a natural killer cell, are combined. The respective effective ingredients contained in the combination of the present invention may be administered, to a subject, in a simultaneous manner, or in a separate manner at different times. Thus, the respective effective ingredients need not necessarily be present in the combination as a form of a single composition at the time of administration. The combination according to the present invention is related to a combination, a medicament, and a pharmaceutical composition. In embodiments, the components of the combination are administered at a time where both components (drugs) are active in the patient at the same time. It is implied by “synergism” that both drugs are active in the patient at the same time. In embodiments, the components of the combination are administered together, simultaneously, separately or subsequently, either physically or in time. In embodiments, the components of the combination are administered simultaneously.

The pharmaceutical combination is applicable to all types of tumors, including solid cancers and blood cancers. Unlike the blood cancers, the solid cancers refer to cancers formed in a lump in organs. Cancers developed in most of organs correspond to the solid cancers. There is no particular limitation on tumors that can be treated using the pharmaceutical combination according to the present invention. The pharmaceutical combination according to the present invention can have synergistic therapeutic effects, for example, on at least one disease selected from the group consisting of gastric cancer, liver cancer, lung cancer, colorectal cancer, breast cancer, prostate cancer, ovarian cancer, pancreatic cancer, cervical cancer, thyroid cancer, laryngeal cancer, acute myeloid leukemia, brain tumor, neuroblastoma, retinoblastoma, head and neck cancer, salivary gland cancer, and lymphoma.

As used herein, the terms “synergy”, “synergistic effect”, and “synergistic action” mean going beyond an additive effect of a combination which is expected due to combination.

Further, the lymphoma may be, specifically, a malignant tumor of B cell origin, which may be at least one selected from the group consisting of non-Hodgkin's lymphoma, chronic lymphocytic leukemia, and acute lymphoblastic leukemia.

The non-Hodgkin's lymphoma may be a lymphoma selected from the group consisting of follicular lymphoma, small lymphocytic lymphoma, mucosa-associated lymphoid tissue, marginal zone, diffuse large B cells, Burkitt, and mantle cells.

Two components of synergistic combination of the present invention, for example, an antibody specific for CD19 and an NK cell, may be administered in a concerted, simultaneous, separate, or sequential manner, from a physical or temporal point of view. As used herein, the term “administered” or “administering” is intended to encompass, but not limited to, deliveries by an injectable form such as by an intravenous, intramuscular, intradermal, or subcutaneous route, or by a mucosal route, for example, as a nasal spray or aerosol for inhalation, or as an ingestible solution, capsule, or tablet. In addition, the pharmaceutical combination may be administered in combination with another drug or physiologically active substance of which therapeutic effects are known for a disease to be treated, or may be formulated in the form of a combined preparation with another drug.

For example, the NK cell may be administered before and/or separately with administration of the antibody specific to CD19, an example of which is MOR208. In a case where the two components are administered together, these components may be formulated together in one pharmaceutical composition which may contain a pharmaceutically acceptable carrier or excipient. Alternatively, the two components may also be formulated in different pharmaceutical compositions. In this case, the two components may be administered in a simultaneous or separate manner. Specifically, in a case where the antibody specific for CD19 and the NK cell are administered, these components may be simultaneously active in the body of a patient having received the administration. For example, in a case where MOR208 is administered weekly and the NK cell is administered daily, active ingredients of both drugs are simultaneously present in the patient.

Further, another aspect of the present invention provides a kit for the treatment of cancer, comprising the above-described combination. Types of the kit are not particularly limited, and a kit having a type commonly used in the art can be used.

The kit may be packaged in a form in which the antibody specific for CD19 and the NK cell as described above are contained in individual containers, respectively, or in a form in which the antibody specific for CD19 and the NK cell as described above are contained in a single container which is divided into one or more compartments. Each of the antibody specific for CD19 and the NK cell may be packaged in a unit dose form with a single dose, but not limited thereto. The antibody specific for CD19 and the NK cell in the kit may be administered in combination, separately at appropriate times, depending on a health condition or the like of a subject to receive the administration.

Another aspect of the present invention provides a use of the above-described pharmaceutical combination for preparing a medicament for the treatment of cancer. In addition, there is provided a use of the above-described pharmaceutical combination for the treatment of cancerous diseases and similar pathologies.

Further, still another aspect of the present invention provides a method for the treatment of cancer, comprising step (a) of preparing an NK cell, step (b) of preparing an antibody specific for CD19, comprising the HCDR1 region of SYVMH (SEQ ID NO: 1), the HCDR2 region of NPYNDG (SEQ ID NO: 2), the HCDR3 region of GTYYYGTRVFDY (SEQ ID NO: 3), the LCDR1 region of RSSKSLQNVNGNTYLY (SEQ ID NO: 4), the LCDR2 region of RMSNLNS (SEQ ID NO: 5), and the LCDR3 region of MQHLEYPIT (SEQ ID NO: 6), and step (c) of administering, to a subject, therapeutically effective amounts of the antibody specific for CD19 and the NK cell in combination.

As used herein, the term “subject” means a mammal including a human, having a cancerous disease in a condition which can be alleviated, suppressed, or treated by administering the antibody specific for CD19 and the NK cell according to an embodiment of the present invention, or suffering from such a disease. Further, the subject may be a non-human animal, and the term “non-human animal” includes vertebrates such as mammals and non-mammals, for example, primates except humans, sheep, dogs, cats, horses, cows, chickens, amphibians, reptiles, etc.

It is possible to provide a method for the treatment of cancer, comprising step (a) of preparing an NK cell, step (b) of preparing an antibody specific for CD19, and step (c) of administering, to a subject, therapeutically effective amounts of the antibody specific for CD19 and the NK cell in combination.

Since the step (a) of preparing the NK cell, and the antibody specific to CD19 in the step (b) have already been described above, descriptions thereof are omitted in order to avoid excessive redundancy.

The step (c) of administering may be carried out by administering the antibody specific for CD19 and the NK cell in combination, in a simultaneous, sequential, or reverse-order manner.

An aspect of the present disclosure comprises a synergistic combination of an antibody specific for CD19 comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), a LCDR1 region of sequence RSSKSLQNVNGNTYLY (SEQ ID NO: 4), a LCDR2 region of sequence RMSNLNS (SEQ ID NO: 5), and a LCDR3 region of sequence MQHLEYPIT (SEQ ID NO: 6) and an NK cell for the treatment of non-Hodgkin's lymphoma, chronic lymphocytic leukemia and/or acute lymphoblastic leukemia. In embodiments, the non-Hodgkin's lymphoma is selected from the group consisting of follicular lymphoma, small lymphocytic lymphoma, mucosa-associated lymphoid tissue, marginal zone, diffuse large B cell, Burkitt's, and mantle cell. In an embodiment the NK cell is MG4101.

An aspect of the present disclosure comprises an antibody specific for CD19 comprising an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), a LCDR1 region of sequence RSSKSLQNVNGNTYLY (SEQ ID NO: 4), a LCDR2 region of sequence RMSNLNS (SEQ ID NO: 5), and a LCDR3 region of sequence MQHLEYPIT (SEQ ID NO: 6) for the treatment of non-Hodgkin's lymphoma, chronic lymphocytic leukemia and/or acute lymphoblastic leukemia, wherein said antibody specific for CD19 is used in combination with an NK cell. In embodiments, the non-Hodgkin's lymphoma is selected from the group consisting of follicular lymphoma, small lymphocytic lymphoma, mucosa-associated lymphoid tissue, marginal zone, diffuse large B cell, Burkitt's, and mantle cell. In an embodiment, the NK cell is MG4101. In embodiments, the non-Hodgkin's lymphoma is follicular lymphoma. In embodiments, the non-Hodgkin's lymphoma is small lymphocytic lymphoma. In embodiments, the non-Hodgkin's lymphoma is mucosa-associated lymphoid tissue. In embodiments, the non-Hodgkin's lymphoma is marginal zone lymphoma. In embodiments, the non-Hodgkin's lymphoma is diffuse large B cell lymphoma. In embodiments, the non-Hodgkin's lymphoma is Burkitt's lymphoma. In embodiments, the non-Hodgkin's lymphoma is mantle cell lymphoma.

Another aspect comprises a method of treating non-Hodgkin's lymphoma, chronic lymphocytic leukemia and/or acute lymphoblastic leukemia in an individual in need thereof, which method comprises administration of an antibody specific for CD19 in combination with an NK cell. In embodiments of the method, the antibody specific for CD19 comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6). In embodiments of the method, the antibody comprises the exemplified antibody specific for CD19. In embodiments of the method the NK cell is MG4101.

Another aspect includes a use of an antibody specific for CD19 wherein said antibody comprises an HCDR1 region of sequence SYVMH (SEQ ID NO: 1), an HCDR2 region of sequence NPYNDG (SEQ ID NO: 2), an HCDR3 region of sequence GTYYYGTRVFDY (SEQ ID NO: 3), an LCDR1 region of sequence RSSKSLQNVNGNTYLY (SEQ ID NO: 4), an LCDR2 region of sequence RMSNLNS (SEQ ID NO: 5), and an LCDR3 region of sequence MQHLEYPIT (SEQ ID NO: 6) in the manufacture of a medicament for the treatment of non-Hodgkin's lymphoma, chronic lymphocytic leukemia and/or acute lymphoblastic leukemia in synergistic combination with an NK cell. In an embodiment, the NK cell is MG4101.

Further, route of administration, dosage, and frequency of administration of the antibody specific for CD19 and the NK cell may vary depending on a patient's condition and presence or absence of side effects, and thus, administration may be done to a subject in various ways and amounts. Optimal method of administration, dosage, and frequency of administration can be selected by a person skilled in the art within suitable ranges.

The antibody specific CD19 and the NK cell may be administered parenterally, and the administration can be done by any method suitable for intratumoral, intraperitoneal, subcutaneous, intradermal, intranodal, and intravenous routes, and the like. Specifically, the antibody specific for CD19 and the NK cell may be administered intratumorally, intraperitoneally, or intravenously. On the other hand, dosages of the antibody specific for CD19 and the NK cell can be determined depending on schedule of administration, dosage, and a health condition of a patient.

Further, the NK cell may be administered two to five times, and may be administered to a subject at intervals of 1 to 30 days, 2 to 15 days, or 3 to 10 days.

The antibody specific for CD19 may be administered within 2 to 48 hours after administration of the NK cell. Specifically, the antibody specific for CD19 may be administered continuously for 3 to 5 days before administration of the NK cell, and for 9 to 11 days, once a day, starting from 2 hours after administration of the NK cell. Details related to cancer that is a target disease on which synergistic therapeutic effects are exhibited due to the above combined administration are as described in the pharmaceutical combination.

Another aspect of the present invention provides a use of the antibody specific for CD19 used in a pharmaceutical combination with the natural killer cell for preparing a medicament for treatment of cancer.

Since the antibody specific for CD19, the natural killer cell and the combined administration thereof used in the present invention have already been described above, descriptions thereof are omitted in order to avoid excessive redundancy.

Hereinafter, the present invention will be described in more detail by way of the following examples. Here, the present inventors intend to describe synergistic effects of combined administration of the anti-CD19 antibody and the natural killer cell. However, the following examples are given to merely illustrate the present invention, and a scope of the present invention is not limited thereto only.

TABLE 1

| | | SEQ ID | |
|---|---|---|---|
| Antibody | | NO: | [aa]/DNA |
| MOR208 | HCDR1 | SEQ ID | SYVMH |
| | | NO: 1 | |
| | HCDR2 | SEQ ID | NPYNDG |
| | | NO: 2 | |
| | HCDR3 | SEQ ID | GTYYYGTRVFDY |
| | | NO: 3 | |
| | LCDR1 | SEQ ID | RSSKSLQNVNGNTYL |
| | | NO: 4 | Y |
| | LCDR2 | SEQ ID | RMSNLNS |
| | | NO: 5 | |
| | LCDR3 | SEQ ID | MQHLEYPIT |
| | | NO: 6 | |
| | VH | SEQ ID | EVQLVESGGGLVKPG |
| | | NO: 8 | GSLKLSCAASGYTFT |
| | | | SYVMHWVRQAPGKGL |
| | | | EWIGYINPYNDGTKY |
| | | | NEKFQGRVTISSDKS |
| | | | ISTAYMELSSLRSED |
| | | | TAMYYCARGTYYYGT |
| | | | RVFDYWGQGTLVTVS |
| | | | S |
| | VL | SEQ ID | DIVMTQSPATLSLSP |
| | | NO: 9 | GERATLSCRSSKSLQ |
| | | | NVNGNTYLYWFQQKP |
| | | | GQSPQLLIYRMSNLN |
| | | | SGVPDRFSGSGSGTE |
| | | | FTLTISSLEPEDFAV |
| | | | YYCMQHLEYPITFGA |
| | | | GTKLEIK |
| | Heavy | SEQ ID | ASTKGPSVFPLAPSS |
| | chain | NO: 10 | KSTSGGTAALGCLVK |
| | constant | | DYFPEPVTVSWNSGA |
| | region | | LTSGVHTFPAVLQSS |
| | | | GLYSLSSVVTVPSSS |
| | | | LGTQTYICNVNHKPS |
| | | | NTKVDKKVEPKSCDK |
| | | | THTCPPCPAPELLGG |
| | | | PDVFLFPPKPKDTLM |
| | | | ISRTPEVTCVVVDVS |
| | | | HEDPEVQFNWYVDGV |
| | | | EVHNAKTKPREEQFN |
| | | | STFRVVSVLTVVHQD |
| | | | WLNGKEYKCKVSNKA |
| | | | LPAPEEKTISKTKGQ |
| | | | PREPQVYTLPPSREE |
| | | | MTKNQVSLTCLVKGF |
| | | | YPSDIAVEWESNGQP |
| | | | ENNYKTTPPMLDSDG |
| | | | SFFLYSKLTVDKSRW |
| | | | QQGNVFSCSVMHEAL |
| | | | HNHYTQKSLSLSPGK |
| | Light | SEQ ID | RTVAAPSVFIFPPSD |
| | chain | NO: 11 | EQLKSGTASVVCLLN |
| | constant | | NFYPREAKVQWKVDN |
| | region | | ALQSGNSQESVTEQD |
| | | | SKDSTYSLSSTLTLS |
| | | | KADYEKHKVYACEVT |
| | | | HQGLSSPVTKSFNRG |
| | | | EC |
| CD19 | Human | SEQ ID | MPPPRLLFFLLFLTP |
| | CD19 | NO: 7 | MEVRPEEPLVVKVEE |
| | | | GDNAVLQCLKGTSDG |
| | | | PTQQLTWSRESPLKP |
| | | | FLKLSLGLPGLGIHM |
| | | | RPLAIWLFIFNVSQQ |
| | | | MGGFYLCQPGPPSEK |
| | | | AWQPGWTVNVEGSGE |
| | | | LFRWNVSDLGGLGCG |
| | | | LKNRSSEGPSSPSGK |
| | | | LMSPKLYVWAKDRPE |
| | | | IWEGEPPCLPPRDSL |
| | | | NQSLSQDLTMAPGST |
| | | | LWLSCGVPPDSVSRG |
| | | | PLSWTHVHPKGPKSL |
| | | | LSLELKDDRPARDMW |
| | | | VMETGLLLPRATAQD |
| | | | AGKYYCHRGNLTMSF |
| | | | HLEITARPVLWHWLL |
| | | | RTGGWKVSAVTLAYLI |
| | | | FCLCSLVGILHLQRA |
| | | | LVLRRKRKRMTDPTR |
| | | | RFFKVTPPPGSGPQN |
| | | | QYGNVLSLPTPTSGL |
| | | | GRAQRWAAGLGGTAP |
| | | | SYGNPSSDVQADGAL |
| | | | GSRSPPGVGPEEEEG |
| | | | EGYEEPDSEEDSEFY |
| | | | ENDSNLGQDQLSQDG |
| | | | SGYENPEDEPLGPED |
| | | | EDSFSNAESYENEDE |
| | | | ELTQPVARTMDFLSP |
| | | | HGSAWDPSREATSLG |
| | | | SQSYEDMRGILYAAP |
| | | | QLRSIRGQPGPNHEE |
| | | | DADSYENMDNPDGPD |
| | | | PAWGGGGRMGTWSTR |

### Preparation Example 1. Cell Lines, Primary Cells, and Culture Conditions

Raji and Ramos cell lines (human Burkitt's lymphoma cell lines) were obtained from the American Type Culture Collection (ATCC, USA). Raj and Ramos cell lines were maintained in RPMI 1640 medium supplemented with 10% fetal bovine serum (FBS) and 2 mmol/L of glutamine. Regarding NK cells, peripheral blood mononuclear cells (PBMCs) were isolated randomly from healthy donors, and NK cells were expansion-cultured under good manufacturing practice (GMP) conditions in a manner as described above (MG4101, Green Cross Labcell Corporation). Briefly, PBMCs in which CD3+ T cells were almost completely removed were expansion-cultured in a CellGro SCGM serum-free medium (Cellgenix) containing 1% auto-plasma, 5×10⁶ cells/mL of autologous PBMC irradiated with radiation (2,000 rad), 10 ng/mL of monoclonal antibody for CD3 (OKT3, eBioscience), and 500 IU/mL of IL2 (Proleukin). 500 IU/mL of IL2 was supplied to the medium every 2 to 3 days while the NK cells were cultured.

### Preparation Example 2. Monoclonal Antibody

MOR208, which is an antibody specific for CD19, was provided by MorphoSys AG (Germany). Rituximab (anti-CD20 antibody) was purchased from Roche. Antigen expression level determined as MOR208 or rituximab antibodies bound per cell were determined by flow cytometry (Quantibrite kit, BD Biosciences) according to the manufacturer's instructions. The number of MOR208 binding sites per cell of Raji and Ramos are 56,702 and 27,772 respectively. The number of rituximab binding sites per cell of Raji and Ramos are 59,974 and 168,253, respectively.

### Preparation Example 3. Calcein-Release Cytotoxicity Assay

Target cells were labeled with 30 uM calcein-acetoxymethyl ester (calcein-AM; Molecular Probes) at 37° C. for 1 hour. After washing, the labeled target cells were dispensed into a 96-well plate at 1×10⁴ cells per well. A monoclonal antibody (MOR208, rituximab, or control irrelevant antibody) was added to the target cells (at a concentration between 0.001 ng/mL and 10 μg/mL). MG4101 was harvested, washed and added to the target cells in a 3:1 (E/T) ratio. After 2 hours, the plate was spun at 2,000 rpm for 3 minutes, and 100 μL of the supernatant was collected. Then, a fluorescence value (OD480/535 nm) was measured using a fluorescent microplate reader (Victor3, Perkin Elmer), to determine an amount of calcein released. A specific amount of calcein released was calculated using the following equation:

Specific dissolution %=(Test release−Spontaneous release)×100/(Maximum release−Spontaneous release)

The maximum dissolution was achieved with 1% Triton X-100.

### Preparation Example 4. Mouse Tumor Model

### Preparation Example 4.1. Establishment of Ramos Xenograft Model in SCID Mice

Ramos cells (1×10⁵, 5×10⁵, or 10×10⁵ per mouse) were suspended in PBS and intravenously injected into SCID mice. Animals were monitored daily for signs of disease, hind limb paralysis, or death.

### Preparation Example 4.2. MOR208 Dose Test in Ramos Xenograft Model

Ramos cells (1×10⁶ per mouse) were suspended in PBS and intravenously injected into SCID mice (day 0). MOR208 was administered by intravenous injection at various concentrations (20, 60, or 200 ug/100 uL/head) on days 3, 6, 10, 13, 17, and 20. Animals were monitored daily for signs of disease, hind limb paralysis, or death.

### Preparation Example 4.3. MG4101 Dose Test in Ramos Xenograft Model

Ramos cells (1×10⁶ per mouse) were suspended in PBS and intravenously injected into SCID mice (day 0). MG4101 was administered by intravenous injection at various concentrations (1, 2, or 5×10⁷/400 uL/head) on days 4, 7, 11, 14, 18, and 21. Animals were monitored daily for signs of disease, hind limb paralysis, or death.

### Preparation Example 4.4. MG4101+MOR208 Efficacy Test in Ramos Xenograft Model

Ramos cells (1×10⁶ per mouse) were suspended in PBS and intravenously injected into SCID mice (day 0). Mice were divided into 6 groups and adjusted as follows:
i) Freezing medium and intravenous injection of hIgG (200 ug)
ii) Intravenous injection of MG4101 (2×10⁷/400 uL/head)
iii) Intravenous injection of MOR208 (60 ug/head)
iv) Intravenous injection of MOR208 (200 ug/head)
v) Intravenous injection of MG4101 (2×10⁷/400 uL/head) and intravenous injection of MOR208 (60 ug/head)
vi) Intravenous injection of MG4101 (2×10⁷/400 uL/head) and intravenous injection of MOR208 (200 ug/head).

These administrations were carried out by intravenous injection twice a week for 3 weeks with hIgG or MOR208 at various concentrations (60 or 200 ug/100 uL/head) on days 3, 6, 10, 13, 17, and 20.

Intravenous injection of MG4101 (at a concentration of 2×10⁷/400 uL/head) was carried out on days 4, 7, 11, 14, 18, and 21. Animals were monitored daily for signs of disease, hind limb paralysis, or death.

### Experimental Example 1. Confirmation of Strong In Vitro ADCC Activity of MOR208 Against Malignant B Cells

Because NK cell-mediated ADCC is very important for activity of monoclonal antibodies, the present inventors firstly determined ADCC activity of MOR208 (anti-CD19 antibody) and MG4101 (NK cell) in Raji and Ramos which are Burkitt's lymphoma cell lines, as compared with rituximab which is a standard monoclonal antibody for the treatment of lymphoma, and is used as a positive control and reference value in the present invention.

First, Raji and Ramos cell lines (Burkitt's lymphomas, CD19+CD20+) were cultured for 2 hours with increasing concentrations of MOR208 or rituximab in the presence of activated and expanded liquid-type and frozen-type MG4101 obtained from three healthy donors (E/T ratio: 3:1).

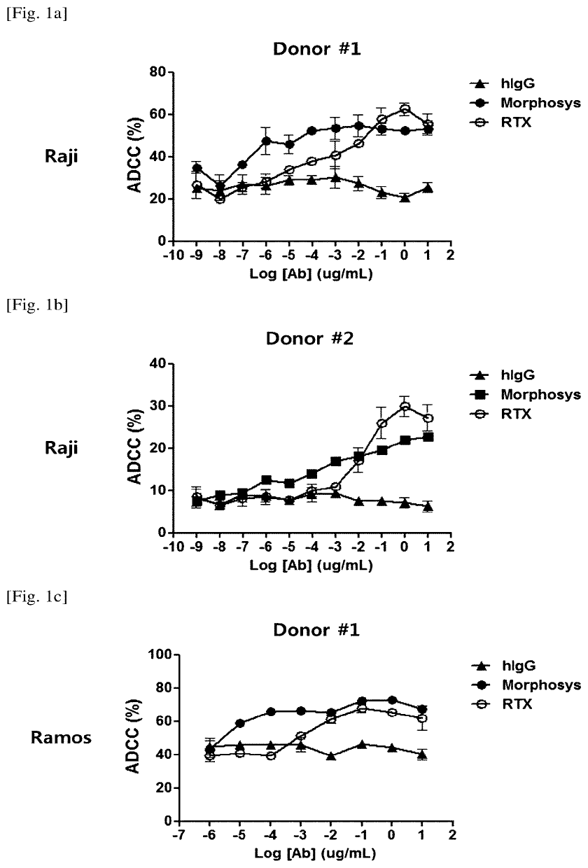

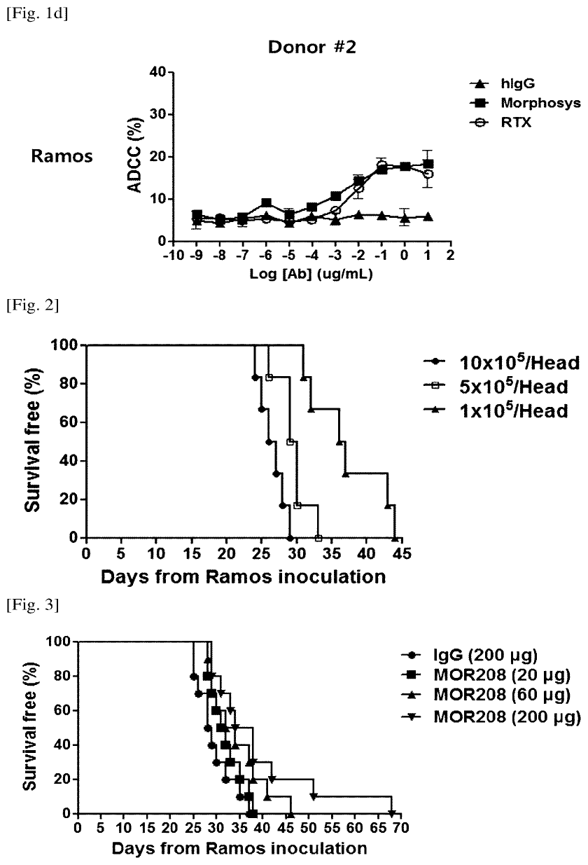

A specific ADCC (%) was calculated by the following equation, and the results are shown in FIG. 1 (in which graphs show average ADCC (%)±SD of 3 experimental values obtained from one NK cell donor representative in two different donors).

(Sample release−Spontaneous release)/(Maximum release−Spontaneous release)×100.

As shown in FIGS. 1a to 1d, MOR208 exhibited remarkable in vitro ADCC activity against Raji and Ramos cell lines. As compared with a hIgG co-treated control, both MOR208 and rituximab showed a dose dependent efficacy against both tumor cell lines with activity already at low antibody concentrations and an increased maximum tumor cell lysis. Taking it into consideration that MOR208 and rituximab showed a comparable maximum killing efficacy as well as substantially higher levels of CD20 compared to CD19 (material and methods), MOR208 has been demonstrated to have an overall lower EC₅₀ compared to rituximab on various malignant B cells that include Raji, and Ramos cells. This indicates that MOR208 can be an interesting alternative therapeutic agent under a condition where rituximab exhibits poor effects.

### Experimental Example 2. Effects of MOR208 on Lymphoma Cell Death in Xenograft SCID Mice

### Experimental Example 2.1. Establishment of Ramos Xenograft Model in SCID Mice Model

With respect to MOR208 in vivo experiments, a xenograft SCID mice model was established using Ramos cells to evaluate suitability of Ramos cell lines. Groups of 6 SCID mice were injected i.v. with 1×10⁵, 5×10⁵, 10×10⁵ Ramos cells or PBS and monitored signs of illness daily. All mice died between day 26 and day 37 after injection of Ramos.

Referring to FIG. 2, intravenous injection with various concentrations of Ramos cells showed tumorigenic activity in mice which is characterized by symptoms including progressive weight loss and hind limb paralysis, and nearly 100% mice died between day 26 and day 37 after injection. For follow-up experiments, an optimal dose of the Ramos cell line was selected at 1×10⁶ cells.

### Experimental Example 2.2. MOR208 Dose Test in Ramos Xenograft Model

An efficacy of MOR208 on B lymphoma cell death is clinically important for a possibility of depleting primary malignant B cells in patients. The present inventors intravenously injected Ramos cells into SCID mice and evaluated an antitumor activity of MOR208 in a propagated lymphoma model in which MOR208 was administered as described in the “Preparation Examples” above. In this model, disseminated Ramos cells infiltrated all mouse organs including the central nervous system, thus reflecting diseases such as propagated Burkitt's lymphoma and acute lymphoblastic leukemia. In this regard, groups of 10 SCID mice were injected i.v. with 1×10⁶ Ramos cells (day 0). Mice were treated with monoclonal antibodies (20, 60, or 200 μg/100 L/head) or hIgG (hIgG 200 μg/100 μL/head) as a negative control on days 3, 6, 10, 13, 17, and 20, and observed daily for signs of disease.

As shown in FIG. 3, treatment with 200 μg of MOR208 significantly prolonged survival of mice (P<0.001) as compared with animals treated with the control monoclonal antibody. A median survival time in mice treated with 200 μg of MOR208, which was 36 days, was found to be longer than that in mice treated with control antibody, which was 28.5 days.

### Experimental Example 2.3. MG4101 Dose Test in Ramos Xenograft Model

SCID mice injected with Ramos cells were intravenously injected with in vitro expanded and activated MG4101 cells of healthy donors as described in the “Preparation Examples”. This MG4101 cell supplement is intended to mimic presence of tissue NK cells which may be present in a patient's organ. Specifically, groups of 10 SCID mice were injected i.v. with 1×10⁶ Ramos cells (day 0). Mice were administered with MG4101 (1, 2, or 5×10⁷/400 μL/head) or as a control with the NK cell freezing medium alone on days 4, 7, 11, 14, 18, and 21, and observed daily for signs of disease.

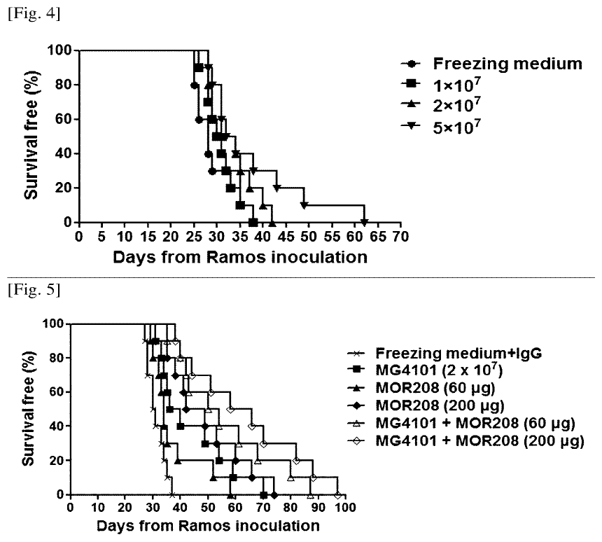

As shown in FIG. 4, a survival rate of mice to which MG4101 was administered at a dosage of 5×10⁷ cells per mouse was significantly enhanced (P<0.05), as compared with mice treated with the control (freezing medium). The median survival time of mice treated with 5×10⁷ cells of MG4101 was 33 days, and that of mice treated with the freezing medium control was 28 days.

### Experimental Example 2.4. Confirmation of Effects Due to Combined Administration of MG4101+MOR208 in Ramos Xenograft Model

Mice injected with Ramos cells were randomly divided into several groups, and treated with two dosages of MOR208 or MG4101 alone, two dosages of a combination of MOR208 and MG4101, or NK cell freezing medium containing hIgG as a control on day 3 after inoculation of Ramos cells as described in the “Preparation Examples”. Specifically, groups of 10 SCID mice were injected i.v. with 1×10⁶ Ramos cells (day 0). Mice were treated with monoclonal antibodies (60 or 200 μg/100 μL/head) on days 3, 6, 10, 13, 17, and 20. Mice were intravenously injected with MG4101 (at a concentration of 2×10⁷/400 μL/head) on days 4, 7, 11, 14, 18, and 21, and monitored daily.

As shown in FIG. 5, it was confirmed that the mice treated with MOR208 exhibited significantly enhanced protective effects as compared with the injected control hIgG in freezing medium. MG4101-injected groups exhibited significant improvement as compared to the control. The median survival time was increased to 45.5 and 38 days for the 200 μg of MOR208-treated mice (P<0.0001) and the 2×10⁷ cells of MG4101-treated mice (P<0.001) respectively compared with 30.5 days for the control freezing medium-treated ones.

On the other hand, animals treated with the combination of MOR208 and MG4101 exhibited a synergistic increase in survival during the experiment period. Specifically, the median survival time was increased to 52 and 62 days for the 60 μg and 200 μg of MOR208-treated mice respectively on the back ground of the 2×10⁷ cells of MG4101 co-injection (P<0.0001). Considerable synergistic effects of the combined administration of MOR208 and MG4101 were also exhibited in an analysis for increased survival. In particular, each increase in the mean survival rate due to individual treatment with MOR208 or MG4101 was 11.5% (60 μg of MOR208), 49.2% (200 μg of MOR208), and 24.6% (2×10⁷ cells of MG4101), respectively. In contrast, simultaneous treatment of MOR208 and MG4101 showed effects of increased survival rate of 70.5% (60 μg of MOR208) and 103.3% (200 μg of MOR208), respectively, in a situation where MG4101 was simultaneously injected. These results confirm that, as compared with animals injected separately with MOR208 or MG4101, the simultaneous treatment (combination) of MG4101 and MOR208 significantly prolonged survival of mice, in particular, by a factor of 2 in a case where 60 μg of MOR208 was simultaneously injected.

In other words, both the in vitro and in vivo data identified in the present invention suggest possibilities that a combination of an anti-CD19 antibody and a natural killer cell, that is, combination therapy of the two components, can be an effective therapeutic agent for human B lymphoma, leukemia, and the like, and also exhibit remarkable therapeutic effects on various B cell malignant tumors.

1.-27. (canceled)

28. A method for treatment of diffuse large B-cell lymphoma in a human subject in need thereof, comprising administering to the human subject an antibody specific for CD19 and an NK cell, wherein the antibody comprises a heavy chain variable region comprising an HCDR1 region of SYVMH (SEQ ID NO:1), an HCDR2 region of NPYNDG (SEQ ID NO:2), and an HCDR3 region of GTYYYGTRVFDY (SEQ ID NO:3), and wherein the antibody comprises a light chain variable region comprising an LCDR1 region of RSSKSLQNVNGNTYLY (SEQ ID NO:4), an LCDR2 region of RMSNLNS (SEQ ID NO:5), and an LCDR3 region of MQHLEYPIT (SEQ ID NO:6).

29. The method according to claim 28, wherein the NK cell is prepared by the following steps (i) to (iii):
step (i) of performing a stationary culture of isolated NK cells in a culture medium containing anti-CD3 antibodies, cytokines, and feeder cells to stimulate intercellular contacts;
step (ii) of adding cytokines, anti-CD3 antibodies, and feeder cells thereto for re-stimulation and performing a stationary culture again to induce intracellular contacts; and
step (iii) of adding a medium containing cytokines to the cells and performing a stationary or suspension culture while constantly maintaining a concentration of the cells and a concentration of the cytokines.

30. The method according to claim 28, wherein the NK cell is prepared by a method comprising co-culturing feeder cells and seed cells, wherein the feeder cells are CD4(+) T cells selected from the group consisting of CD4(+) T cells that have been isolated ex vivo, CD4(+) T cells that have been expansion-cultured ex vivo, and CD4(+) T cell lines.

31. The method according to claim 30, wherein the CD4(+) T cell lines are CD4(+)/CD1(−) T cell lines.

32. The method according to claim 31, wherein the CD4(+)/CD1(−) T cell lines are H9 or HuT78 cell lines.

33. The method according to claim 30, wherein the seed cells are one or more selected from the group consisting of peripheral blood-derived cells, peripheral blood leukocyte cells, peripheral blood mononuclear cells (PBMCs), umbilical cord blood leukocyte cells, umbilical cord blood mononuclear cells, natural killer cells derived from stem cells, enriched natural killer cells, and isolated natural killer cells.

34. The method according to claim 28, wherein the antibody specific for CD19 and the NK cell are administered in a simultaneous manner.

35. The method according to claim 28, wherein the antibody specific for CD19 and the NK cell are administered in a separate manner.

36. The method according to claim 28, wherein the heavy chain variable region comprises the sequence EVQLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:8) and the light chain variable region comprises the sequence DIVMTQSPATLSLSPGERATLSCRSSKSLQNVNGNTYLYWFQQKPGQSPQLLIYRMSNL NSGVPDRFSGSGSGTEFTLTISSLEPEDFAVYYCMQHLEYPITFGAGTKLEIK (SEQ ID NO:9).

37. The method according to claim 36, wherein the antibody comprises a heavy chain constant region of ASTKGPSVFPLAPSSKSTSGGTAALGCLVKDYFPEPVTVSWNSGALTSGVHTFPAVLQSS GLYSLSSVVTVPSSSLGTQTYICNVNNHKPSNTKVDKKVEPKSCDKTHTCPPCPAPELLGG PDVFLFPPKPKDTLMISRTPEVTCVVVDVSSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:10) and a light chain constant region of RTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO:11).

38. A method for treatment of follicular lymphoma in a human subject in need thereof, comprising administering to the human subject an antibody specific for CD19 and an NK cell, wherein the antibody comprises a heavy chain variable region comprising an HCDR1 region of SYVMH (SEQ ID NO:1), an HCDR2 region of NPYNDG (SEQ ID NO:2), and an HCDR3 region of GTYYYGTRVFDY (SEQ ID NO:3), and wherein the antibody comprises a light chain variable region comprising an LCDR1 region of RSSKSLQNVNGNTYLY (SEQ ID NO:4), an LCDR2 region of RMSNLNS (SEQ ID NO:5), and an LCDR3 region of MQHLEYPIT (SEQ ID NO:6).

39. The method according to claim 38, wherein the NK cell is prepared by the following steps (i) to (iii):
step (i) of performing a stationary culture of isolated NK cells in a culture medium containing anti-CD3 antibodies, cytokines, and feeder cells to stimulate intercellular contacts;
step (ii) of adding cytokines, anti-CD3 antibodies, and feeder cells thereto for re-stimulation and performing a stationary culture again to induce intracellular contacts; and
step (iii) of adding a medium containing cytokines to the cells and performing a stationary or suspension culture while constantly maintaining a concentration of the cells and a concentration of the cytokines.

40. The method according to claim 38, wherein the NK cell is prepared by a method comprising co-culturing feeder cells and seed cells, wherein the feeder cells are CD4(+) T cells selected from the group consisting of CD4(+) T cells that have been isolated ex vivo, CD4(+) T cells that have been expansion-cultured ex vivo, and CD4(+) T cell lines.

41. The method according to claim 40, wherein the CD4(+) T cell lines are CD4(+)/CD1(−) T cell lines.

42. The method according to claim 41, wherein the CD4(+)/CD1(−) T cell lines are H9 or HuT78 cell lines.

43. The method according to claim 40, wherein the seed cells are one or more selected from the group consisting of peripheral blood-derived cells, peripheral blood leukocyte cells, peripheral blood mononuclear cells (PBMCs), umbilical cord blood leukocyte cells, umbilical cord blood mononuclear cells, natural killer cells derived from stem cells, enriched natural killer cells, and isolated natural killer cells.

44. The method according to claim 38, wherein the antibody specific for CD19 and the NK cell are administered in a simultaneous manner.

45. The method according to claim 38, wherein the antibody specific for CD19 and the NK cell are administered in a separate manner.

46. The method according to claim 38, wherein the heavy chain variable region comprises the sequence EVQLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:8) and the light chain variable region comprises the sequence DIVMTQSPATLSLSPGERATLSCRSSKSLQNVNGNTYLYWFQQKPGQSPQLLIYRMSNL NSGVPDRFSGSGSGTEFTLTISSLEPEDFAVYYCMQHLEYPITFGAGTKLEIK (SEQ ID NO:9).

47. The method according to claim 46, wherein the antibody comprises a heavy chain constant region of ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:10) and a light chain constant region of RTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO:11).

48. A method for treatment of marginal zone lymphoma in a human subject in need thereof, comprising administering to the human subject an antibody specific for CD19 and an NK cell, wherein the antibody comprises a heavy chain variable region comprising an HCDR1 region of SYVMH (SEQ ID NO:1), an HCDR2 region of NPYNDG (SEQ ID NO:2), and an HCDR3 region of GTYYYGTRVFDY (SEQ ID NO:3), and wherein the antibody comprises a light chain variable region comprising an LCDR1 region of RSSKSLQNVNGNTYLY (SEQ ID NO:4), an LCDR2 region of RMSNLNS (SEQ ID NO:5), and an LCDR3 region of MQHLEYPIT (SEQ ID NO:6).

49. The method according to claim 48, wherein the NK cell is prepared by the following steps (i) to (iii):
step (i) of performing a stationary culture of isolated NK cells in a culture medium containing anti-CD3 antibodies, cytokines, and feeder cells to stimulate intercellular contacts;
step (ii) of adding cytokines, anti-CD3 antibodies, and feeder cells thereto for re-stimulation and performing a stationary culture again to induce intracellular contacts; and
step (iii) of adding a medium containing cytokines to the cells and performing a stationary or suspension culture while constantly maintaining a concentration of the cells and a concentration of the cytokines.

50. The method according to claim 48, wherein the NK cell is prepared by a method comprising co-culturing feeder cells and seed cells, wherein the feeder cells are CD4(+) T cells selected from the group consisting of CD4(+) T cells that have been isolated ex vivo, CD4(+) T cells that have been expansion-cultured ex vivo, and CD4(+) T cell lines.

51. The method according to claim 50, wherein the CD4(+) T cell lines are CD4(+)/CD1(−) T cell lines.

52. The method according to claim 51, wherein the CD4(+)/CD1(−) T cell lines are H9 or HuT78 cell lines.

53. The method according to claim 50, wherein the seed cells are one or more selected from the group consisting of peripheral blood-derived cells, peripheral blood leukocyte cells, peripheral blood mononuclear cells (PBMCs), umbilical cord blood leukocyte cells, umbilical cord blood mononuclear cells, natural killer cells derived from stem cells, enriched natural killer cells, and isolated natural killer cells.

54. The method according to claim 48, wherein the antibody specific for CD19 and the NK cell are administered in a simultaneous manner.

55. The method according to claim 48, wherein the antibody specific for CD19 and the NK cell are administered in a separate manner.

56. The method according to claim 48, wherein the heavy chain variable region comprises the sequence EVQLVESGGGLVKPGGSLKLSCAASGYTFTSYVMHWVRQAPGKGLEWIGYINPYNDGT KYNEKFQGRVTISSDKSISTAYMELSSLRSEDTAMYYCARGTYYYGTRVFDYWGQGTL VTVSS (SEQ ID NO:8) and the light chain variable region comprises the sequence DIVMTQSPATLSLSPGERATLSCRSSKSLQNVNGNTYLYWFQQKPGQSPQLLIYRMSNL NSGVPDRFSGSGSGTEFTLTISSLEPEDFAVYYCMQHLEYPITFGAGTKLEIK (SEQ ID NO:9).

57. The method according to claim 56, wherein the antibody comprises a heavy chain constant region of ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSSHEDPEVQFNWYVDGVEVHNAKTKPREEQF NSTFRVVSVLTVVHQDWLNGKEYKCKVSNKALPAPEEKTISKTKGQPREPQVYTLPPSR EEMTKNQVSLTCLVKGFYPSDIAVEWESNGQPENNYKTTPPMLDSDGSFFLYSKLTVDK SRWQQGNVFSCSVMHEALHNHYTQKSLSLSPGK (SEQ ID NO:10) and a light chain constant region of RTVAAPSVFIFPPSDEQLKSGTASVVCLLNNFYPREAKVQWKVDNALQSGNSQESVTEQ DSKDSTYSLSSTLTLSKADYEKHKVYACEVTHQGLSSPVTKSFNRGEC (SEQ ID NO:11).


SEQUENCE LISTING (11 sequences)

SEQ ID NO: 1 - protein, 5 aa, synthetic construct
SYVMH

SEQ ID NO: 2 - protein, 6 aa, synthetic construct
NPYNDG

SEQ ID NO: 3 - protein, 12 aa, synthetic construct
GTYYYGTRVFDY

SEQ ID NO: 4 - protein, 16 aa, synthetic construct
RSSKSLQNVNGNTYLY

SEQ ID NO: 5 - protein, 7 aa, synthetic construct
RMSNLNS

SEQ ID NO: 6 - protein, 9 aa, synthetic construct
MQHLEYPIT

SEQ ID NO: 7 - protein, 556 aa, synthetic construct [sequence omitted]

SEQ ID NO: 8 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 9 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 10 - protein, 329 aa, synthetic construct
ASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 107 aa, synthetic construct [sequence omitted]